SEQUENCE LISTING (10 sequences)

SEQ ID NO: 1 - DNA, 133 bp, synthetic construct
  source 1..133: mol_type = other DNA
ggtaaatgtgagcactcacaattcattttgcaaaagttgttgactttatctacaaggtgtggcataatgtgtgtaattgtgagcggataacaattaagcttagtcgacagctagcacataaggaggaactact

SEQ ID NO: 2 - DNA, 169 bp, synthetic construct
  source 1..169: mol_type = other DNA
gaaaagaatgatgtaagcgtgaaaaattttttaaaaaatctcttgacattggaagggagatatgttattataagaattgcggaattgtgagcggataacaattcataattgtgagcggataacaattcaaccccaaaggaggtgggatccgtgatagcggtaccctcga

SEQ ID NO: 3 - protein, 238 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 4 - protein, 238 aa, synthetic construct
MGKGEELFTGVVPILVELDGDVNGHKFSVSGEGEGDATYGKLTLKFICTTGKLPVPWPTLVTTLTYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 5 - protein, 10 aa, synthetic construct
GGGGSGGGGS

SEQ ID NO: 6 - protein, 12 aa, synthetic construct
LEGGSGGSGGSG

SEQ ID NO: 7 - protein, 10 aa, synthetic construct
GGSGGSGGGS

SEQ ID NO: 8 - protein, 376 aa, synthetic construct
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 9 - DNA, 1357 bp, synthetic construct
  source 1..1357: mol_type = other DNA
cggtggaaacgaggtcatcatttccttccgaaaaaacggttgcatttaaatcttacatatgtaatactttcaaagactacatttgtaagatttgatgtttgagtcggctgaaagatcgtacgtaccaattattgtttcgtgattgttcaagccataacactgtagggatagtggaaagagtgcttcatctggttacgatcaatcaaatattcaaacggagggagacgattttgatgatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaaacggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagtgacaaaaaagcggggaatccccgctttttttacgcc

SEQ ID NO: 10 - protein, 360 aa, synthetic construct
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKTAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

(19) United States
(12) Patent Application Publication
(10) Pub. No.: US 2026/0234689 A1
(43) Pub. Date: Aug. 13, 2026

(54) CONSTRUCTION AND USE OF A BIOSENSOR SELECTION SYSTEM FOR LACTOSE OR ANALOGUE THEREOF IN BACILLUS SUBTILIS

(71) Applicant: JIANGNAN UNIVERSITY, Wuxi (CN)

(72) Inventors: Long LIU, Wuxi (CN); Jian CHEN, Wuxi (CN); Xueqin LV, Wuxi (CN); Guocheng DU, Wuxi (CN); Xianhao XU, Wuxi (CN); Jianghua LI, Wuxi (CN); Yanfeng LIU, Wuxi (CN); Quanwei ZHANG, Wuxi (CN)

(21) Appl. No.: 19/403,590

(22) Filed: Nov. 28, 2025

Related U.S. Application Data
(63) Continuation of application No. PCT/CN2025/101714, filed on Jun. 18, 2025.

(30) Foreign Application Priority Data
May 16, 2024 (CN) 202410607555.6

(51) Int. Cl. C12Q 1/02; C07K 14/195; C12N 1/16; C12N 15/75; C12Q 1/6897; C12R 1/125

(52) U.S. Cl. CPC C12Q 1/025; C07K 14/195; C12N 1/16; C12N 15/75; C12Q 1/6897; C12R 2001/125

(57) ABSTRACT

The invention provides the construction and use of a biosensor screening system for lactose or an analog thereof in Bacillus subtilis. The present disclosure designs a “dual-selection” gene circuit for screening biosensor repressor proteins based on lactose operons and applicable to Bacillus subtilis. The DNA-binding activity of the repressor proteins is screened via a lethal circuit under negative selection, and the allosteric activation activity screening is achieved based on a fluorescence signal under positive selection. Ultimately, biosensors with recognition activity for target lactose analogs are obtained. A novel biosensor responsive to substances such as lactose, 2′-fucosyllactose, and 3-fucosyllactose can be obtained by using the screening platform, and it has been demonstrated that the biosensor constructed based on the screening results in the present disclosure exhibits relatively high detection sensitivity and specificity.

This application is a Continuation of PCT/CN2025/101714, filed on Jun. 18, 2025, which claims priority to Chinese Patent Application No. 202410607555.6, filed on May 16, 2024, which is incorporated by reference for all purposes as if fully set forth herein.

A Sequence Listing XML file named “10015_0190_Sequence_Listing.xml” created on Nov. 28, 2025 and having a size of 12,371 bytes, is filed concurrently with the specification. The sequence listing contained in the XML file is part of the specification and is herein incorporated by reference in its entirety.

### FIELD OF THE INVENTION

The present disclosure relates to the construction and use of a biosensor screening system for lactose or an analog thereof in Bacillus subtilis, and pertains to the technical field of synthetic biology and metabolic engineering.

### DESCRIPTION OF THE RELATED ART

In 1961, Monord and Jacob from Institut Pasteur in France found that Escherichia coli could determine the production of enzymes involved in lactose metabolism depending on the presence or absence of glucose and lactose in the environment. Through investigation of the above phenomenon, they proposed the concepts of operons and operator genes. After the lactose operon concept was put forward, researchers successively identified various operons, including the tryptophan operon, the histidine operon, the arabinose operon, and the like. The gene regulatory function of operons enables microorganisms to activate or suppress the expression of specific genes in response to environmental changes, ensuring that microorganisms can rapidly synthesize necessary enzymes and metabolites when specific metabolites are required, while simultaneously halting the synthesis of other kinds of metabolites. In the face of rapidly changing and complex external environments, this regulatory mechanism is indispensable for living organisms, as the presence of operons enables organisms to have stronger adaptability in variable external environments.

Using various operon elements as models, researchers have developed a variety of metabolite biosensors. When the target metabolite is present, the metabolite biosensor can achieve responsiveness by altering the conformation of proteins and can convert the target metabolite concentration signal into a fluorescence signal, a growth rate signal, a metabolic pathway signal, etc. In recent years, metabolite biosensors have played an important role in the construction of microbial cell factories. Meanwhile, as the application scope expands, the complex application conditions have imposed higher demands on the metabolite recognition ranges, molecular response levels, signal output intensities, etc., of metabolite biosensors. However, most biosensors currently in use have been developed primarily as natural metabolite biosensors based on naturally occurring biological elements that respond to specific metabolites. As a result, natural metabolite biosensors fail to adapt to the demands for responding to a variety of novel metabolites.

To broaden the application scope of metabolite biosensors, it is necessary to change the response target of the natural metabolite sensing element. In general, modifying the inducer specificity of these biosensor-responsive proteins is difficult, as such modifications may disrupt their original allosteric characteristics, ultimately impairing their binding effect to specific genetic elements and leading to failure of the entire system. Therefore, to increase the response range of biosensors to respond to different types of metabolites or inducers and enable broader application, it is necessary to develop a rapid, accurate, and efficient screening platform for the screening of biosensors responsive to target compounds.

### SUMMARY OF THE INVENTION

To address the above issues, the present disclosure provides a method for constructing a screening platform for a biosensor for lactose or an analog thereof in Bacillus subtilis and use thereof. The screening platform consists of an expression cassette of DNA-binding transcriptional repressor LacI, a screening system gene circuit expression cassette (an IPTG inducible promoter, a reporter gene, and a suicide gene), and gene expression elements such as promoters, RBSs, and terminators required for the expression of the above genes. The above screening platform can be constructed in a plasmid vector and transformed into the cytoplasm of Bacillus subtilis for use or integrated into the genome of Bacillus subtilis for use, to enable the screening of the biosensor or the element thereof.

A first objective of the present disclosure is to provide a screening method for a biosensor for lactose or an analog thereof, comprising steps of:
S1, constructing a mutant library of DNA-binding transcriptional repressor LacI;
S2, constructing a recombinant cell library, where each recombinant cell in the cell library comprises a gene expression cassette of a mutant of DNA-binding transcriptional repressor LacI and a screening system gene circuit expression cassette, where the screening system gene circuit expression cassette comprises an inducible promoter, a reporter gene, and a suicide gene; S3, negative selection: culturing the recombinant cells of the recombinant cell library obtained in S2 to allow expression of genes in the expression cassette of the mutant of DNA-binding transcriptional repressor LacI and the screening system gene circuit expression cassette, then transferring the recombinant cell library to a first culture medium for cultivation, and screening for surviving cells, where the first culture medium comprises a non-cytotoxic prodrug, and a protein encoded by the suicide gene is capable of converting the non-cytotoxic prodrug into a cytotoxic active drug; and
S4, positive selection: transferring the surviving cells from S3 to a second culture medium for cultivation, and screening the cells based on signal intensities of the reporter gene to obtain a target biosensor, where the second culture medium comprises lactose or an analog thereof. Further, the host cells of the recombinant cell library may be suitable microbial cells selected based on the type of the biosensor, such as common Bacillus subtilis and Escherichia coli. Further, in step S1, the construction method for the mutant library includes saturation mutagenesis and/or random mutagenesis.

Further, in step S2, the gene expression cassette of the mutant of DNA-binding transcriptional repressor LacI is any DNA sequence capable of being transcribed and translated to produce the mutant of DNA-binding transcriptional repressor LacI, and not only comprises the encoding gene of the LacI mutant, but also comprises expression elements such as a promoter, a RBS, and a terminator required for the expression of the gene. For example, the sequence of the expression cassette comprises but is not limited to the sequence shown in SEQ ID NO. 9.

Further, the promoter that drives the expression of the gene of the mutant of DNA-binding transcriptional repressor LacI is preferably a constitutive promoter, and may be specifically selected based on the host cell, preferably to achieve low expression or non-leaky expression in the target host (most preferably non-leaky expression).

Further, in step S2, the screening system gene circuit expression cassette is integrated or retained episomally, and the gene expression cassette of the mutant of DNA-binding transcriptional repressor LacI is retained episomally.

Further, in step S2, the reporter gene may be any gene whose encoded product signal can be detected directly or indirectly, and the type of the reporter gene includes, but is not limited to, those emitting fluorescence without a substrate (such as a fluorescent protein gene), those interacting with radioactive or fluorescent substrates (such as luciferase), and the like.

Further, the fluorescent protein gene includes: a green fluorescent protein gene, a yellow fluorescent protein gene, a red fluorescent protein gene, a deep red fluorescent protein gene, a near-infrared fluorescent protein gene, an orange fluorescent protein gene, a cyan fluorescent protein gene, a blue fluorescent protein gene, a deep blue fluorescent protein gene, and the like, as well as genes of mutants of the above fluorescent proteins or fluorescent protein genes derived by modification, for example, sfGFP having an amino acid sequence shown in SEQ ID NO. 3 or eGFP having an amino acid sequence shown in SEQ ID NO. 4.

Further, in step S2, the suicide gene may be a thymidine kinase gene (hsvTK), a cytosine deaminase gene (CD), or the like. Correspondingly, the non-cytotoxic prodrug selected in step S3 is 5-fluoro-2′-deoxyuridine (5FdU), 5-fluorocytosine (5-FC), or the like. 5FdU is converted into a toxic substance, 5-fluoro-2′-deoxyuridine-5′-monophosphate (5FdUMP), under the action of thymidine kinase; 5-FC is metabolized into 5-fluorouracil by CD in microorganisms, causing cell death.

Further, in step S2, the fluorescent protein gene is linked to the herpes simplex virus thymidine kinase (hsvTK) gene via a flexible linker; that is, the screening platform gene circuit expression cassette is composed of an inducible promoter, and a tandem fusion of a reporter gene, a linker, and a suicide gene, which is controlled by the inducible promoter. The amino acid sequence of the flexible linker may be any one of those shown in SEQ ID NO. 5, SEQ ID NO. 6, and SEQ ID NO. 7.

Further, in step S3, the non-cytotoxic prodrug is added to the first culture medium at a concentration of 1 nM to 1000 mM.

Further, in step S4, the lactose analog includes, but is not limited to, 2′-fucosyllactose (2′-FL), 3-fucosyllactose (3-FL), and the like.

Further, in step S4, the lactose or the analog thereof is added to the second culture medium at a concentration of 1 nM to 1000 mM.

Further, in step S4, screening the cells based on the signal intensities of the reporter gene is as follows: when the reporter gene is a fluorescent protein gene, cells exhibiting relatively high fluorescence intensities (as long as there is a fluorescence signal, with the original or higher signal intensity being preferred) are selected as the target cellular biosensor.

Further, in step S4, the method for screening the cells includes, but is not limited to, a flow cytometry screening method, a microfluidic screening method, a plate spreading screening method, a liquid culture medium-based screening method, and the like.

Further, after step S4, if necessary, a method for sequencing the LacI mutant in the target cell is further included as a means of preparing biosensor elements.

A second objective of the present disclosure is to provide a screening system for a biosensor for lactose or an analog thereof. The screening system comprises:
a recombinant cell library, wherein each recombinant cell in the cell library comprises a gene expression cassette of a mutant of DNA-binding transcriptional repressor LacI and a screening system gene circuit expression cassette, where the screening system gene circuit expression cassette comprises an inducible promoter, a reporter gene, and a suicide gene;
a first culture medium, where the first culture medium comprises a non-cytotoxic prodrug, and a protein encoded by the suicide gene is capable of converting the non-cytotoxic prodrug into a cytotoxic active drug; certainly, those skilled in the art know that when the screening system gene circuit expression cassette is retained episomally, the first culture medium further comprises an antibiotic corresponding to a resistance gene of a plasmid vector used; and a second culture medium, where the second culture medium comprises lactose or an analog thereof; similarly, when the screening system gene circuit expression cassette is retained episomally, the second culture medium also comprises an antibiotic corresponding to the resistance gene of the plasmid vector used.

A third objective of the present disclosure is to provide use of the above screening system in screening a biosensor for lactose or an analog thereof or an element used in the biosensor.

A fourth objective of the present disclosure is to provide a Bacillus subtilis biosensor for lactose or an analog thereof. The Bacillus subtilis biosensor comprises: a recombinant Bacillus subtilis comprising a gene expression cassette of a mutant of DNA-binding transcriptional repressor LacI, where the mutant of DNA-binding transcriptional repressor LacI has an amino acid residue substitution at any one of the following positions based on the sequence shown in SEQ ID NO. 10:
substitution of alanine for valine at position 150;
substitution of glycine for serine at position 193;
substitution of isoleucine for glutamine at position 291;
substitution of valine for glutamine at position 291;
substitution of histidine for isoleucine at position 79; and
substitution of phenylalanine for glutamine at position 291.

Further, the Bacillus subtilis biosensor further comprises: an inducible promoter, and a reporter gene which is initiated by the promoter, such that the presence or absence of lactose or an analog thereof is detected based on whether the reporter gene is expressed.

Further, in step S2, the inducible promoter is derived from a P_{lac} promoter in the lactose operon, including but not limited to a P_{hy-spank} promoter, a P_{grac100} promoter, and the like. The nucleotide sequences of the P_{hy-spank} promoter and the P_{grac100} promoter are shown in SEQ ID NO. 1 and SEQ ID NO. 2, respectively.

A fifth objective of the present disclosure is to provide a mutant of DNA-binding transcriptional repressor LacI. The mutant of DNA-binding transcriptional repressor LacI has an amino acid residue substitution at any one of the following positions based on the sequence shown in SEQ ID NO. 10:
substitution of alanine for valine at position 150;
substitution of glycine for serine at position 193;
substitution of isoleucine for glutamine at position 291;
substitution of valine for glutamine at position 291;
substitution of histidine for isoleucine at position 79; and
substitution of phenylalanine for glutamine at position 291.

A sixth objective of the present disclosure is to provide a nucleic acid encoding the mutant of DNA-binding transcriptional repressor LacI.

A seventh objective of the present disclosure is to provide an expression vector comprising the nucleic acid.

An eighth objective of the present disclosure is to provide a recombinant cell comprising the nucleic acid. The host cell is preferably a microbial cell, and most preferably Bacillus subtilis. A ninth objective of the present disclosure is to provide use of the Bacillus subtilis biosensor, the mutant of DNA-binding transcriptional repressor LacI, the nucleic acid, the expression vector, or the recombinant cell in detecting lactose or an analog thereof.

The beneficial effects of the present disclosure are as follows:
(1) The present disclosure provides a method for constructing a screening platform for a biosensor for lactose and an analog thereof. Specifically, a mutant library of DNA-binding transcriptional repressor LacI and a screening system gene circuit expression cassette comprising an inducible promoter, a reporter gene, and a suicide gene are transformed into cells, 5-fluoro-2′-deoxyuridine (5FdU) is first added into the culture medium, and the screening for mutants of DNA-binding transcriptional repressor LacI having promoter sequence-binding activity is achieved based on whether the cells survive. Subsequently, specific lactose analogs (lactose, 2′-FL, 3-FL, etc.) are added to the culture medium, and the screening for mutants of DNA-binding transcriptional repressor LacI having binding ability to specific lactose analogs is achieved based on the signal of the reporter gene. The screening platform is simple to construct and achieves high-throughput screening of the mutant library of DNA-binding transcriptional repressor LacI to obtain mutants of DNA-binding transcriptional repressor LacI that can respond to specific lactose analogs.
(2) By means of the self-constructed screening platform, the present disclosure identifies LacI mutants having strong binding activity to the inducible promoter and high sensitivity to lactose and an analog thereof, such as V150A, S193G, 179H, Q291I, Q291V, and Q291F. These mutants exhibit strong binding to the promoter in the absence of lactose or an analog thereof, thereby repressing transcription. However, in the presence of the test substance, the mutants respond sensitively and dissociate from the promoter, and the reporter gene initiates expression to achieve detection. It has been demonstrated that these mutants exhibit significantly improved detection performance compared to the wild-type LacI and possess specificity.

### BRIEF DESCRIPTION OF THE DRAWINGS

FIG. 1 is a schematic diagram of a gene circuit of a screening platform for a lactose analog biosensor in Bacillus subtilis and flowchart of screening for a mutant for a lactose analog biosensor according to the present disclosure.

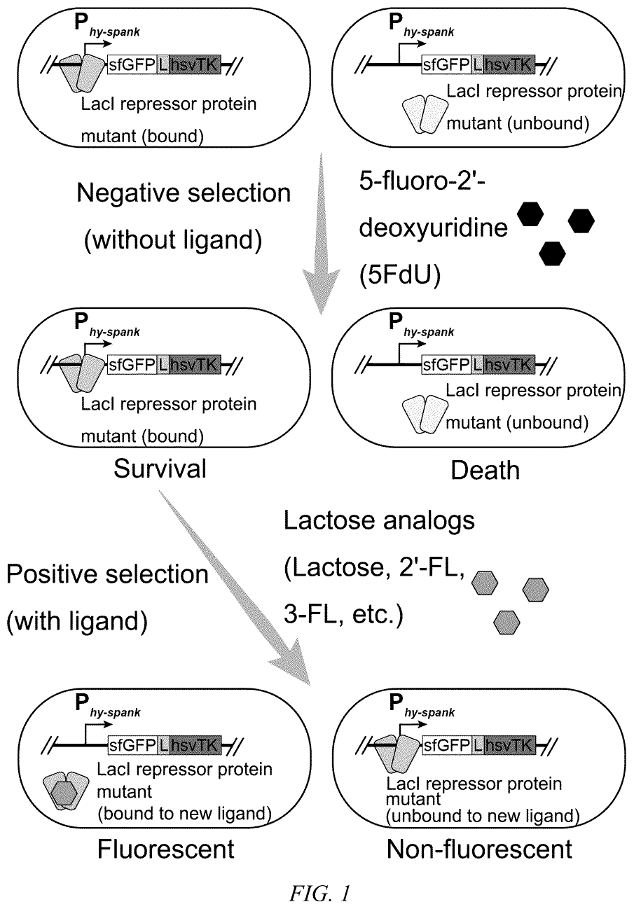

FIG. 2 illustrates response curves of the mutants for lactose.

### DETAILED DESCRIPTION OF THE PREFERRED EMBODIMENTS

The present disclosure will be further described below with reference to the drawings and specific embodiments, such that those skilled in the art can better understand and implement the present disclosure. However, the embodiments should not be construed as limiting the present disclosure.

The materials involved in the following examples are as follows:
liquid LB culture medium (g/L): peptone 10, yeast powder 5, and NaCl 10.

The primer sequences involved in the following examples are as follows:

TABLE 1
Primer sequence

| Primer | |
|---|---|
| name | Primer sequence |
| F1 | cgaaacaataattggtacgtacgatctttcagccg |
| | actc |
| R1 | ctcctttgctcatagtagttcctccttatgtgcta |
| | g |
| F2 | ggaggaactactatgagcaaaggagaagaactttt |
| | cac |
| R2 | accaccaccaccagaaccaccaccacctttgtaga |
| | gctcatccatgcc |
| F3 | gtggtggttctggtggtggtggttctatggcttct |
| | tatcctggtcatc |
| R3 | ctgcagttaattagcttcacccatttctctagc |
| F4 | gggtgaagctaattaactgcaggtcgacgtcc |
| R4 | ctgaaagatcgtacgtaccaattattgtttcgtga |
| | ttgttcaagcc |
| F5 | gtgccagctgcattaatgaatcggccaacgcgcg |
| R5 | ctggttacgatcaatcaaatattcaaacggaggga |
| | gacgattttgatgatg |
| F6 | cttagttagcttggccagtgcctaccatcattgat |
| | ggtttctttcggtaagt |
| R6 | ctgaaagatcgtacgtaccaattattgtttcgtcg |
| | acatggatgagcgatgatg |
| F7 | gaaatgggtgaagctaattaactgcagaattctgc |
| | gtgacatccc |
| R7 | tctgatctgccgttcgtaacaggatgtttgaattt |
| | ccgtttaaagaatgggct |

### Example 1. Construction of Screening Platform for Biosensor for Lactose in Bacillus subtilis

The plasmid screening platform for a biosensor for lactose in Bacillus subtilis in this example consisted of a DNA-binding transcriptional repressor LacI expression cassette, an IPTG inducible promoter, an sfGFP fluorescent protein expression gene, a fusion protein linker Linker1, a herpes simplex virus thymidine kinase gene, and gene expression elements such as promoters, RBSs, and terminators required for the expression of the above genes.
(1) Construction of a screening system gene circuit expression cassette and a DNA-binding transcriptional repressor LacI expression cassette to obtain a biosensor screening platform plasmid:

Primers F1 and R1 were designed by using a P_{hy-spank} promoter and an RBS sequence thereof (with the nucleotide sequence shown in SEQ ID NO. 1) as a template, and a DNA fragment of the P_{hy-spank} promoter and the RBS sequence thereof was obtained by PCR amplification. Primers F2 and R2 were designed by using an sfGFP fluorescent protein DNA sequence (with the amino acid sequence shown in SEQ ID NO. 3) as a template, and a DNA fragment of the sfGFP fluorescent protein was obtained by PCR amplification. Primers F3 and R3 were designed by using a herpes simplex virus thymidine kinase sequence (with the amino acid sequence shown in SEQ ID NO. 8) as a template, and a DNA fragment of the herpes simplex virus thymidine kinase was obtained by PCR amplification. The DNA sequence of the fusion protein linker Linker1 is shown in SEQ ID NO. 5, and due to short sequence length, the sequence was added to primers R2 and F3, respectively. Primers F4 and R4 were designed by using the pHT-LacI plasmid vector sequence as a template, and PCR amplification was performed to obtain a plasmid vector fragment containing the expression cassette sequence of DNA-binding transcriptional repressor LacI.

The above DNA fragments were fused by PCR and transformed into Escherichia coli DH5α to construct a pHT-GLH lactose biosensor screening platform plasmid.
(2) Construction of LacI mutant library (comprising a saturation mutant library and a random mutant library):

A pHT-GLH plasmid was extracted, and saturation mutagenesis primers for DNA-binding transcriptional repressor LacI were designed. The pHT-GLH plasmid was used as a template, and a saturation-mutated fragment of the pHT-GLH plasmid was obtained by PCR and transformed into Escherichia coli DH5α to construct a saturation mutant library of DNA-binding transcriptional repressor LacI. The Escherichia coli DH5α containing the saturation mutant library of DNA-binding transcriptional repressor LacI was scaled up for culture, and the saturation mutant library of DNA-binding transcriptional repressor LacI was extracted. Similarly, random mutagenesis primers F5 and R5 were designed, and a random mutant library of DNA-binding transcriptional repressor LacI was constructed using a random mutagenesis kit. The Escherichia coli DH5α containing the random mutant library of DNA-binding transcriptional repressor LacI was scaled up for culture, and the random mutant library of DNA-binding transcriptional repressor LacI was extracted.
(3) Construction of genetically engineered strains:

Bacillus subtilis competent cells were prepared, and the saturation mutant library or the random mutant library of DNA-binding transcriptional repressor LacI was transformed into the competent Bacillus subtilis cells to obtain a cell library of a lactose biosensor screening platform in Bacillus subtilis.

As described above, the screening system gene circuit expression cassette and the DNA-binding transcriptional repressor LacI expression cassette were constructed on the same plasmid and maintained episomally for gene expression in the host cells to construct a screening cell library, which was used for subsequent experiments. Certainly, those skilled in the art can also select the genomic integration method to construct a lactose biosensor screening platform. The specific procedures were as follows:

The genome-integrated Bacillus subtilis lactose biosensor screening platform consisted of a DNA-binding transcriptional repressor LacI expression plasmid (pHT-lacI plasmid), an IPTG inducible promoter, a fluorescent protein expression gene, a fusion protein linker Linker, a herpes simplex virus thymidine kinase gene, and gene expression elements such as promoters, RBSs, and terminators required for the expression of the above genes.

PCR primers F6, R6, F7, and R7 for the upstream homologous arm and the downstream homologous arm used for gene integration were designed by using a Bacillus subtilis amyE integration site DNA sequence as a template, and fragments of the upstream homologous arm and the downstream homologous arm used for gene integration were obtained by PCR amplification. Primers F1 and R1 were designed by using a P_{hy-spank} promoter and an RBS sequence thereof (shown in SEQ ID NO. 1) as a template, and a DNA fragment of the P_{hy-spank} promoter and the RBS sequence thereof was obtained by PCR amplification. Primers F2 and R2 were designed by using an sfGFP fluorescent protein DNA sequence (shown in SEQ ID NO. 3) as a template, primers F3 and R3 were designed by using a herpes simplex virus thymidine kinase sequence (shown in SEQ ID NO. 8) as a template, and a DNA fragment of the herpes simplex virus thymidine kinase was obtained by PCR amplification. The DNA sequence of the fusion protein linker Linker1 is shown in SEQ ID NO. 5, and due to short sequence length, the sequence was added to primers R2 and F3, respectively. Fusion PCR was performed on the above DNA fragments to construct a gene integration fragment. The above gene integration fragment was transformed into Bacillus subtilis 168 for gene integration. After colony growth, colony PCR was performed to verify integration, and Bacillus subtilis strains with successful integration of the fusion fragment were screened. Competent cells of the above successfully integrated Bacillus subtilis strains were then prepared for subsequent use.

A pHT-lacI plasmid (kindly offered by Dr. Li Yang from Jiangnan University) was used as a template, and the saturation mutagenesis primers were subjected to PCR to obtain a saturation-mutated fragment of the pHT-lacI plasmid. The fragment was transformed into Escherichia coli DH5α to construct a saturation mutant library of DNA-binding transcriptional repressor LacI. The Escherichia coli DH5α containing the saturation mutant library of DNA-binding transcriptional repressor LacI was scaled up for culture, and the saturation mutant library of DNA-binding transcriptional repressor LacI was extracted. Similarly, random mutagenesis primers F5 and R5 were designed, and a random mutant library of DNA-binding transcriptional repressor LacI was constructed using a random mutagenesis kit. The Escherichia coli DH5α containing the random mutant library of DNA-binding transcriptional repressor LacI was scaled up for culture, and the random mutant library of DNA-binding transcriptional repressor LacI was extracted.

The saturation mutant library or the random mutant library of DNA-binding transcriptional repressor LacI was transformed into the competent Bacillus subtilis cells with successful integration of the fusion fragment to obtain a cell library of a lactose biosensor screening platform in Bacillus subtilis.

### Example 2. Use of Screening Platform for Biosensor for Lactose in Lactose-Responsive LacI Mutant

This example was intended to screen for LacI mutants having strong binding activity to the promoter and high sensitivity to lactose. The screening included negative selection and positive selection: the binding activity of LacI was screened via a lethal circuit under negative selection, while the allosteric activation activity screening was achieved based on a fluorescence signal under positive selection.

A screening plate containing 5FdU was prepared, and Bacillus subtilis containing the cell library of the lactose biosensor screening platform was spread onto the screening plate for culture. In Bacillus subtilis, the LacI mutants were constitutively expressed, and different mutants exhibited different binding capacities to the promoter P_{hy-spank}, thereby exerting differing degrees of expression repression. The binding of the LacI mutants to the promoter P_{hy-spank} repressed transcription, the herpes simplex virus thymidine kinase lethal gene was not expressed, and the cells survived (when the herpes simplex virus thymidine kinase gene was expressed, the engineered bacterium converted the non-cytotoxic 5FdU precursor in the culture medium into cytotoxic 5FdUMP). Therefore, individual colonies grown on the plate were cells containing the LacI mutants having DNA binding ability obtained through negative selection, and after colony formation, the individual colonies were eluted to prepare bacterial solutions. The cells were eluted using a liquid LB culture medium, and then subjected to liquid shaking culture (37° C., 220 rpm, 10-12 h) in the presence of lactose at a final concentration of 1 mM. The strains harboring lactose-responsive LacI mutants lost the ability to bind to the promoter P_{hy-spank} due to the change in protein configuration after the LacI mutants bound to lactose, and the promoter P_{hy-spank} triggered sfGFP expression to generate a fluorescence signal. Therefore, when the bacterial solution became turbid, flow cytometry screening was performed to obtain cells with an sfGFP fluorescence signal (about 1/10000 to 1/100000 cells with a fluorescence signal), and the obtained cells contained lactose-responsive mutants of DNA-binding transcriptional repressor LacI. The DNA sequence information of the lactose-responsive DNA-binding transcriptional repressor was obtained by subjecting the individual colonies obtained to DNA sequencing (the amino acid sequence of the wild type is shown in SEQ ID NO. 10).

### Example 3. Verification of Lactose Responsiveness

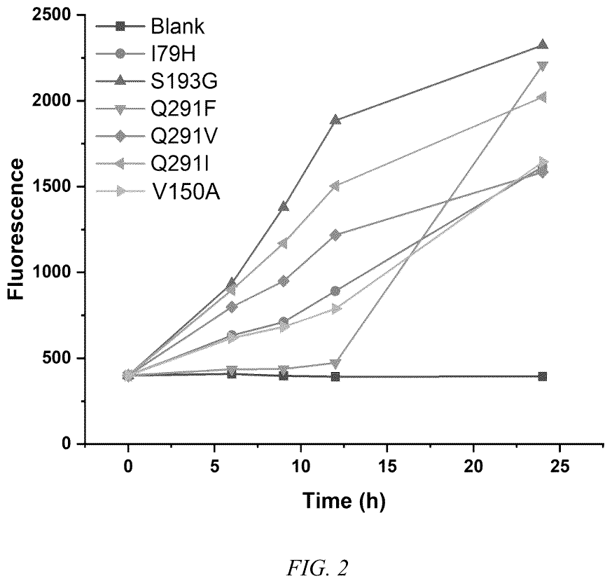

The mutant strains obtained by screening in Example 2 were inoculated into a liquid LB culture medium containing chloramphenicol and cultured at 37° C. and 220 rpm for 10 to 12 h. The fresh seed culture was inoculated into a 96-well plate containing 1 mM lactose and chloramphenicol (inoculum volume: 1%). The 96-well plate was incubated in a plate shaker (37° C., 700 rpm). After 6 h of shaking culture, the fluorescence intensity of the plate was measured every 3 h using a microplate reader, and fluorescence intensity change curves were plotted (as shown in FIG. 2, note: Blank in FIG. 2 represents the control group strain harboring the wild-type lacI repressor protein).

It is obvious that the above examples are merely illustrative for clear illustration and are not intended to limit the embodiments. Various changes and modifications can be made by those of ordinary skill in the art on the basis of the above description. It is unnecessary and impossible to exhaust all the embodiments herein. Obvious changes or modifications derived therefrom still fall within the protection scope of the present disclosure.

1. A screening method for a biosensor for lactose or an analog thereof, comprising steps of:
S1, constructing a mutant library of DNA-binding transcriptional repressor LacI;
S2, constructing a recombinant cell library, wherein each recombinant cell in the cell library comprises a gene expression cassette of a mutant of DNA-binding transcriptional repressor LacI and a gene circuit expression cassette, wherein the gene circuit expression cassette comprises an inducible promoter, a reporter gene, and a suicide gene;
S3, culturing the recombinant cells of the recombinant cell library obtained in S2 to allow expression of genes in the expression cassette of the mutant of DNA-binding transcriptional repressor LacI and the gene circuit expression cassette, then transferring the recombinant cell library to a first culture medium for cultivation, and screening for surviving cells, wherein the first culture medium comprises a non-cytotoxic prodrug, and a protein encoded by the suicide gene is capable of converting the non-cytotoxic prodrug into a cytotoxic active drug; and
S4, transferring the surviving cells from S3 to a second culture medium for cultivation, and screening the cells based on signal intensities of the reporter gene to obtain a target biosensor, wherein the second culture medium comprises lactose or an analog thereof.

2. The screening method according to claim 1, wherein the gene circuit expression cassette is integrated or retained episomally, and the gene expression cassette of the mutant of DNA-binding transcriptional repressor LacI is retained episomally.

3. The screening method according to claim 1, wherein the suicide gene is selected from a thymidine kinase gene or a cytosine deaminase gene, and the non-cytotoxic prodrug is selected from 5-fluorocytosine or 5-fluoro-2′-deoxyuridine.

4. The screening method according to claim 1, wherein the lactose analog at least comprises 2′-fucosyllactose or 3-fucosyllactose.

5. A screening system for a biosensor for lactose or an analog thereof, the screening system comprising:
a recombinant cell library, wherein each recombinant cell in the cell library comprises a gene expression cassette of a mutant of DNA-binding transcriptional repressor LacI and a gene circuit expression cassette, wherein the gene circuit expression cassette comprises an inducible promoter, a reporter gene, and a suicide gene;
a first culture medium, wherein the first culture medium comprises a non-cytotoxic prodrug, and a protein encoded by the suicide gene is capable of converting the non-cytotoxic prodrug into a cytotoxic active drug; and
a second culture medium, wherein the second culture medium comprises lactose or an analog thereof.

6. Use of the screening system according to claim 5 in screening a biosensor or a biosensor element for lactose or an analog thereof.

7. A Bacillus subtilis biosensor for lactose or an analog thereof, the Bacillus subtilis biosensor comprising: a recombinant Bacillus subtilis comprising a gene expression cassette of a mutant of DNA-binding transcriptional repressor LacI, wherein the mutant of DNA-binding transcriptional repressor LacI has an amino acid residue substitution at any one of the following positions based on the sequence shown in SEQ ID NO. 10:
substitution of alanine for valine at position 150;
substitution of glycine for serine at position 193;
substitution of isoleucine for glutamine at position 291;
substitution of valine for glutamine at position 291;
substitution of histidine for isoleucine at position 79; and
substitution of phenylalanine for glutamine at position 291.

8. The Bacillus subtilis biosensor according to claim 7, further comprising: an inducible promoter, and a reporter gene and a suicide gene which are initiated by the promoter.

9. The Bacillus subtilis biosensor according to claim 8, wherein the inducible promoter comprises a Phy-spank promoter or a Pgrac100 promoter.

10. A mutant of DNA-binding transcriptional repressor LacI, wherein the mutant of DNA-binding transcriptional repressor LacI has an amino acid residue substitution at any one of the following positions based on the sequence shown in SEQ ID NO. 10:
substitution of alanine for valine at position 150;
substitution of glycine for serine at position 193;
substitution of isoleucine for glutamine at position 291;
substitution of valine for glutamine at position 291;
substitution of histidine for isoleucine at position 79; and
substitution of phenylalanine for glutamine at position 291.

11. A nucleic acid encoding the mutant of DNA-binding transcriptional repressor LacI according to claim 10.

12. An expression vector comprising the nucleic acid according to claim 11.

13. A recombinant cell comprising the nucleic acid according to claim 11.

14. Use of the Bacillus subtilis biosensor according to claim 7 in detecting lactose or an analog thereof.
